(19) United States
(12) Patent Application Publication
(10) Pub. No.: US 2023/0293118 A1
(43) Pub. Date: Sep. 21, 2023

(54) Technical device and method for generation, recording and identification characteristic pattern in physiological and pathological data for an off-line comparison with previously collected data

(71) Applicant: Hans L. TRINKAUS, Kaiserslautern (DE)

(72) Inventor: Hans L. TRINKAUS, Kaiserslautern (DE)

(21) Appl. No.: 18/059,862

(22) Filed: Nov. 29, 2022

Related U.S. Application Data
(63) Continuation-in-part of application No. 18/000,101, filed on Apr. 26, 2023.

(30) Foreign Application Priority Data
May 29, 2020 (EP) 20177608.5
Nov. 30, 2021 (EP) 10 2021 131 477.1

(51) Int. Cl. A61B 5/00; A61B 5/16

(52) U.S. Cl. CPC A61B 5/742; A61B 5/165; A61B 5/6803

(57) ABSTRACT

An easily manageable device and a method used as an early warning system would be desirable. The device should be usable at virtually any location and operable with little proficiency, and it should be sterilizable, resource-saving and inexpensive. This is achieved by the following: an attachment for the head of a person, the attachment coupled to a first control unit (T) and comprising electrodes (E₁ to E₄) for receiving cerebral electrical activity or brain potentials, wherein the electrodes lead to the first control unit (T). The first control unit (T) has a microprocessor which is capable of recording signals from the electrodes (E₁ to E₄) in a time-related manner via an analog-to-digital converter. The first control unit (T) is coupled to or actuates an actuator element (A₁), which is capable (LDZ₁) of releasing a fragrance in pulses as a stimulus in a flow from at least one pressurized cartridge (K₁). The signals of the electrodes (E₁ to E₄) have an EEG signal level in order to record (FIG. 5, FIG. 6) a group of chemosensorically evoked potentials or at least the chemosensorically-influenced temporal sections thereof in a time-related manner and to analyze them not on the human head.

The present invention deals with a device representing an optimum test environment to generate and acquire reproducible data by means of experiments, and with a method that controls the workflow of the experiments and classifies the resultant data.

The device and the method are to be understood here as closely linked parts of an overall system. The special structural design of the technical device provides environmental conditions which are a prerequisite for successfully conducting experiments that serve to generate experimental data. The latter arise from the reactions of test persons to the steps predetermined by the method. The data acquired by the device are further processed and characterized by the method.

Electroencephalography (EEG), as a method of measurement, records the differences in the electric field potentials emanating from the brain, strictly speaking the potential fluctuations of the dendritic synapses of neural associations located particularly close to the surface (excitatory and inhibitory potentials). It is an examination which is pain-free and harmless for the test person and which can be repeated an arbitrary number of times. The EEG plays an important role in the overall system of the present invention.

What would be desirable is an easy-to-use device and a method that serves as a “Covid19 early warning system”. The device should be usable at virtually any location and operable with little proficiency, and it should be sterilizable, resource-saving, inexpensive, etc.. Preferably, this takes place off line, i.e. not on the human body. Such tests can be carried out elsewhere with a spacing in time and also in location.

The independent claims solve the problem. The dependent claims disclose non-binding but advantageous options.

The invention makes use of the finding that physiological and pathological influences on the generation of a potential lead to changes in the electroencephalogram (EEG). Different degrees of wakefulness change the typical appearance of an EEG, discernible from the altered fundamental frequency patterns. In addition, muscle activity or increased perspiration may cause artifacts and conduction disturbances. In clinical routine, pharmaceuticals, structural cortex changes due to lesions or inflammatory processes, as well as changes in the metabolic conditions and intoxications, by way of example, play a causative role for changes in the EEG.

What is suggested is an attachment for the head of a person, the attachment being coupled to a first control unit. In addition, the attachment has provided thereon electrodes for receiving cerebral electrical activity or brain potentials. The EEG electrodes lead to the first control unit. The first control unit has a microprocessor, which is capable of recording signals from the electrodes in a time-related manner via an analog-to-digital converter. The first control unit is coupled to or actuates an actuator element, which is capable of releasing a fragrance in pulses as a stimulus in a flow from at least one pressurized cartridge. The signals at the electrodes have an EEG signal level in order to record a group of chemosensorically produced potentials or at least the chemosensorically-influenced temporal sections thereof in a time-related manner and to analyze them. In other words, the electrodes are electrodes of the type used when EEG signals are being tapped, and this describes their shape and structure. Preferably, this is not done on the human head, i.e., being an attachment, it is not a diagnostic method practiced on the human body, but it is already a product for use in such a method.

Alternatively, an attachment is coupled to a control unit, virtual-reality glasses and headphones. It comprises electrodes for receiving cerebral electrical activity or brain potentials, the electrodes leading (via a conductive coupling) to the first control unit. The first control unit has a microprocessor that is capable of recording signals from the electrodes in a time-related manner via an analog-to-digital converter. The first control unit is coupled to an actuator element that releases a flow of humidified air from a pressurized first cartridge and is capable of feeding, separately therefrom, a pulsed fragrance as a stimulus. The signals of the electrodes have an EEG signal level or, the other way round, the electrodes are adapted to receive such signals so as to record a group of chemosensorically produced potentials or at least the chemosensorically-influenced temporal sections thereof in a time-related manner. The potentials can be analyzed—preferably not on the human head.

A point of particular importance is the steep rise of the stimulus edge. At least 66% of the stimulus concentration can be reached within 20 msec.

The stimulus duration is, for example, 200 msec, and the stimulus is presented repeatedly, at least 8 times. Preferably at least 10 times. This in a time span of 8 minutes (±10%), so as to obtain in any case a sufficient number of artifact-free measurement results for reliable “averaging” (cf. paragraphs [45] and [52]).

By summing the derived stimulus-synchronous EEG sections, positive and negative fluctuations are added, while random EEG spontaneous activity is averaged out. The event-related potentials can thus be separated from the background noise.

For the most frequently used method of EEG derivation, a unipolar reference lead is used, with surface adhesive electrodes placed on the scalp in a 10/20 distribution.

A distance between adjacent electrode points is 10% or 20% of the total length of an imaginary line from nasion to inion and between the two preauricular points (cf. FIG. 2). Hence, the respective point spacings depend on the individual head length.

The positions on the head surface provide the corresponding designations for the electrodes; frontopolar (Fp), frontal (F), temporal (T), central (C), parietal (P) and occipital (O).

The respective earlobe electrodes (A1 and A2) serve as reference points for the derivation—two grounding electrodes, attached to both sides of the mastoid—so as to avoid interference humming.

The present invention makes use of the finding that the choice of the triggering agent/stimulus is of crucial importance for the generation of chemosensory potentials. It allows to differentiate and interpret the signal obtained. For an olfactory potential, a substance, such as H₂S or PEA, stimulating exclusively and purposefully the olfactory system is used. The trigeminal system can specifically be excited with CO₂.

A device whose development began as early as 1888 by the Dutch physiologist Hendrik Zwaardemaker can be used for a standardized generation of chemosensory stimuli. In the early 1970s, the first measurement systems appeared on the market, these systems being now called “olfactometers”. This tool offers the possibility of embedding a sensory stimulus in a constant, humidified and tempered flow of air. It is thus technically possible to let an odorant flow on and off within time windows of less than 20 ms.

Interstimulation intervals of 30 to 45 seconds are considered to be ideal intervals between stimuli. The flow of air required for presentation should have a flow rate of 7 to 81/min and, because of the concomitant sensitive innervation to be prevented and for better standardizability, it should have a humidity of 70% to 80% and a temperature of 36° C. to 38° C. The entire device or the control unit is controlled via a PC.

In order to allow clinical studies and examinations to be compared with the aid of olfactometry, the test conditions are standardized.

Disturbing influencing factors acting on the test person are minimized. A camera may be installed to check the general status of the test person and to detect physical abnormalities at an early stage, as well as to check the localization of the nosepiece, by way of example. Monitoring through the examiner by means of an additionally provided monitor is possible over the entire period. A curtain placed between the test apparatus and the test person is used for visual stimulus shielding and spatial separation. In order to shield test persons against acoustic interference, such as the clicking of the olfactometer valves during the impulses or background noise in the room, a so-called “white noise” with a volume of at least 80 dB is played to the test persons via headphones.

For stabilizing the vigilance and the level of alertness of the participants over the period of examination, a concentration game is sometimes used. A randomly moving colored rectangle appears on the desktop visible to the test person. The person to be examined is asked to hold a joystick-controlled cursor within the boundaries of this rectangle.

The monitor can be positioned at eye level and at a distance of about 2 m from the seated test person.

This “tracking task” unit serves several functions. Accelerated and sudden eye movements are the main cause of artifacts in EEG derivations. As a result of the slow movement of the tracked object, both keeping the eyes open and gaze stabilization are accomplished. The attention of the test person is standardized by turning to what is happening and a necessary minimal influenceability of the simple game process.

The Robert Koch Institute (RKI) has listed (since May 10, 2020) olfactory and gustatory disturbances as by far the most common symptoms of Covid-19 sufferers. What is special about the new symptoms is that they appear very early. Many infected persons apparently first notice the impairment of the sense of smell and also taste. Only later may other symptoms, such as a scratchy throat and fever or a dry cough, appear as well. Some infected persons do not get any other symptoms at all.

Experts attribute the olfactory and gustatory disturbances to damage to the olfactory cells caused by the coronaviruses. This can be detected early in the measured EEG.

The ENT professional association is even more specific on this point, stating that approximately 85% of the persons suffering from COVID-19 develop olfactory disorders ranging from a reduced ability to smell to a complete loss of smell. In addition, misperceptions of smells often occur. It is reported that “In the case of a corona infection, the areas of the nose responsible for olfactory perception are affected. The cells of the olfactory epithelium and the olfactory bulb are directly affected by the virus and damaged. This receptor failure causes the described olfactory disorders in COVID-19 patients.”.

It is also known that maximum infectivity occurs 1 to 2 days before the onset of the usual symptoms. Hence, it would be possible (and still to be tested) whether EEG abnormalities would already be detectable at this early stage, i.e. before the patient notices symptoms in himself.

### EXAMPLES OF THE INVENTION

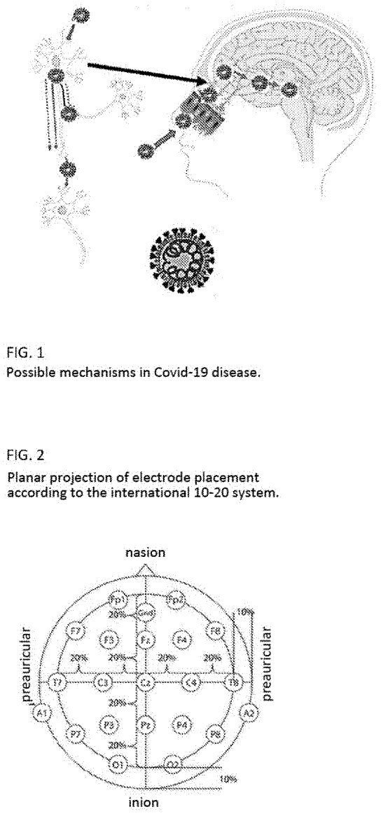

FIG. 1 is an example of the influence of a viral disease on the olfactory organ.

FIG. 2 are reference leads in 10/20 distribution on the attachment.

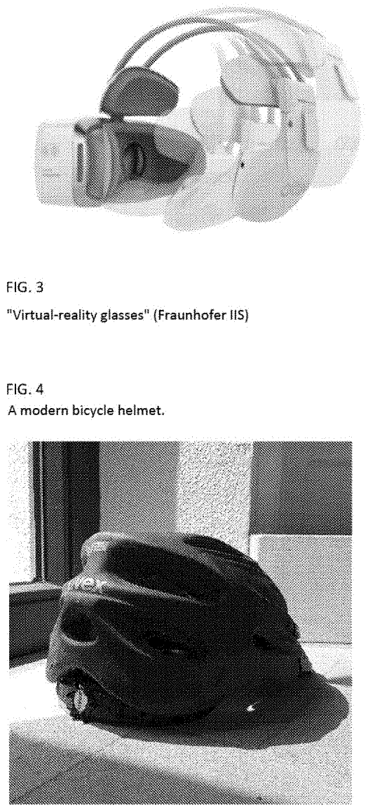

FIG. 3 is an example of virtual-reality glasses.

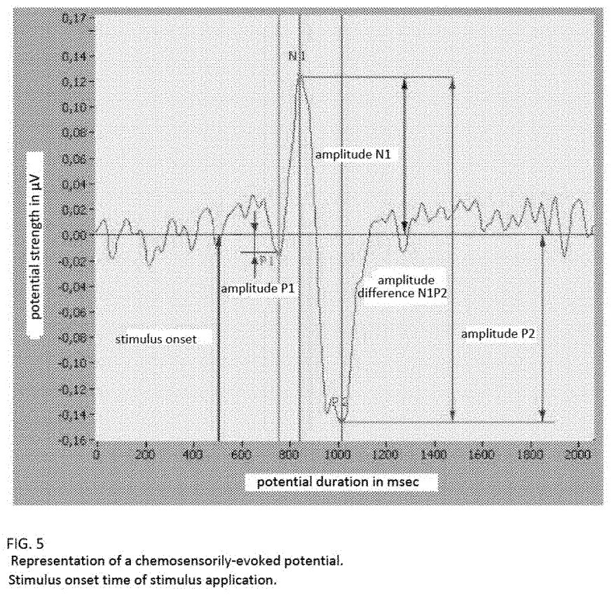

FIG. 4 is a view of a possible attachment with electrodes according to FIG. 2.

FIG. 5 is an example of an EEG with measurement points that may be relevant in chemosensorily evoked potentials.

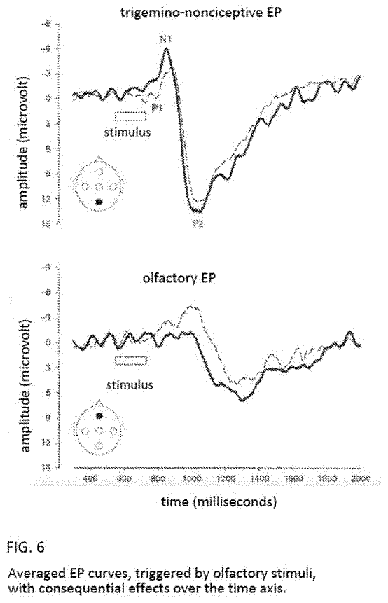

FIG. 6 shows averaged EP curves (EP—evoked potentials) triggered by trigemino-nociceptive as well as olfactory stimuli, with consequential effects over the time axis.

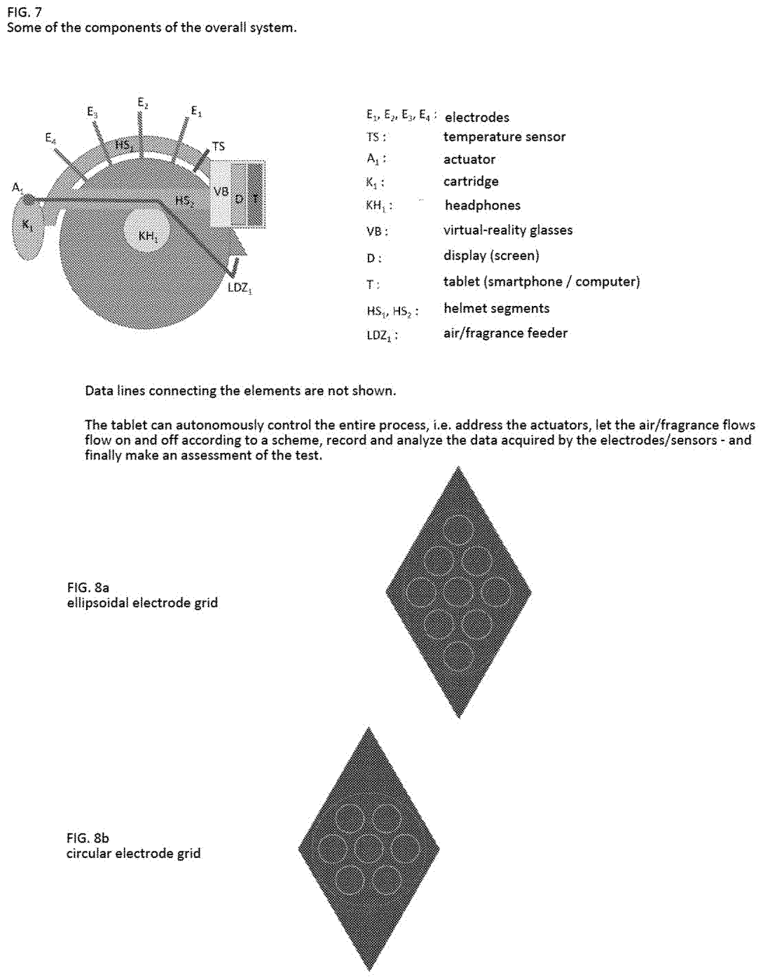

FIG. 7 outlines some of the components of the overall system.

FIG. 8a and FIG. 8b show grid elements with possible placements of the electrodes. The upright diamonds represent, for example, flexible plastic elements that may be incorporated into the helmet segments at the 10/20 positions. The light circles represent possible alternative positions into which an electrode can be inserted or screwed in. A “suitable choice” of the position(s) depends here on a respective individual, real head shape (of the test person). Once an electrode has been inserted or screwed in to such an extent that sufficient contact with the head surface has been established, the pretension and/or flexibility of the plastic element, for example, ensure that the contact between the electrode and the head surface will actually be maintained permanently.

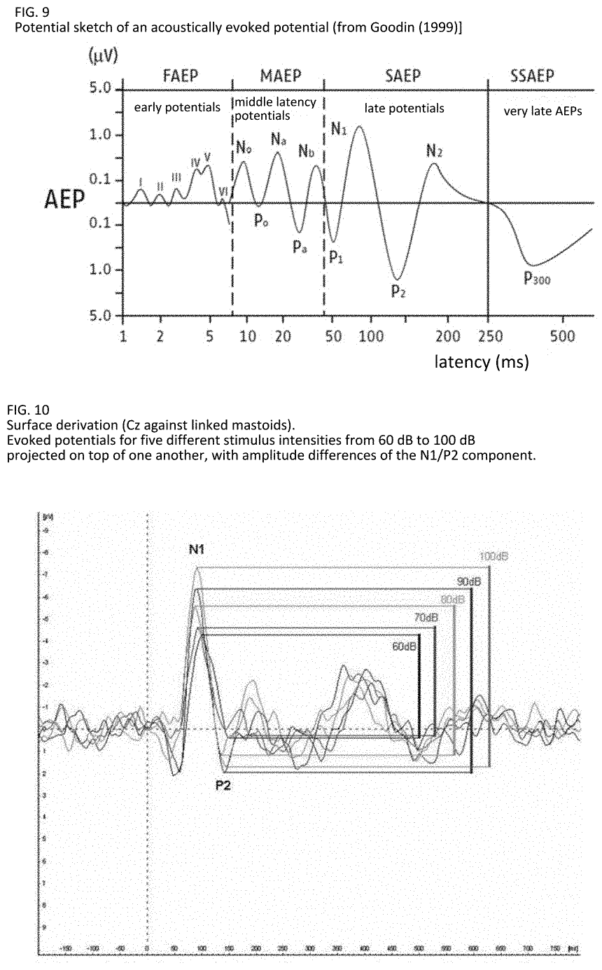

FIG. 9 is a potential sketch of an acoustically evoked potential [from Goodin (1999)].

FIG. 10 shows a surface derivation (Cz against linked mastoids) and evoked potentials for five different stimulus intensities from 60 dB to 100 dB projected on top of one another (for better comparability), with amplitude differences of the N1/P2 component.

In further examples of the present invention, the following is suggested . . .
A cap, a hood, a braid or a special helmet; hereinafter simply: helmet, e.g. similar to a modern bicycle helmet, as an example of an attachment.
The helmet is physically connected by cable or wirelessly by Bluetooth etc. to a control unit or to the WEB—or it works off-line/stand-alone, with software integrated in the helmet.
The helmet may be made of a material that can be re-sterilized at any time with little effort.
The material of the helmet contains electrically conductive components (e.g. wire mesh or sheet metal) for shielding against extrinsic interference fields (Faraday cage).
The helmet is available in several sizes, comparable to shoe sizes; via prefabricated markings, electrodes etc. can be placed/attached with high positional accuracy and also easily. For example, along the apex line Fz, Cz, Pz, on the two earlobes A1, A2 and on the “wink point” Fp2.
The helmet has headphones provided thereon; for informing/instructing/occupying/reassuring the test person. As options.
The helmet has supply lines thereon for supplying a continuous flow of air; in the “inner tube” of the supply line(s), fragrances can be added (program controlled) and supplied to the nasal opening(s). Control and triggering may take place similar to eye pressure measurements, preferably with a tablet or a smartphone or a control element/microprocessor provided in the “virtual-reality-glasses attachment” (see below).
The supply with an air flow/fragrances can be carried out via an (possibly specially simplified/modified) olfactometer or via air/gas cartridges integrated on the helmet, which are attached to the back, by way of example.
The number of cartridges is preferably three. One as an air reservoir under pressure, two other smaller ones containing the odorants mentioned hereinbelow, preferably two different odorants. They are controlled by the first control unit, which is coupled to or actuates one, preferably three, functionally independent actuator elements (together one actuator element). One of the pressurized cartridges supplies humidified air, another one a first fragrance, and the additional one a second fragrance in the flow as a stimulus to the person. Each of the flows is released by a respective actuator element, controlled by the control unit.
The helmet has provided thereon virtual-reality glasses (e.g. similar to the product “Oculus” or to “VR glasses” of Fraunhofer IIS), each with a display, again for informing/instructing/occupying/reassuring the user, the virtual-reality glasses serving, among other things, also as a physical “counterweight” to the air/gas cartridges.
The combination of headphones+display allows ideal test environment conditions (distraction, concentration, avoidance of eye movements, instructions for the user, . . . ).
On the helmet there are also potentials for the earlobes, sensors for measuring the body temperature, the skin resistance, etc.
The helmet has provided thereon a (miniaturized) computer unit, e.g. integrated in the “virtual-reality-glasses attachment”, for example as a tablet or a smartphone; it is used for carrying out the olfactory examination tests/health tests, for controlling the air/fragrance flows, for recording the resultant EEG signals, for storing, outputting/displaying and, possibly, transmitting (via cable or wirelessly) the results.

Methods, as further examples of the present invention.
The software (in the tablet, smartphone or separate microprocessor) allows various test scenarios (sequences of test steps) to be executed.
An analog-to-digital converter picks up the signals and converts them into digital values for the microprocessor. A sampling pattern of a small number of msec may be programmed, or a sampling time that satisfies the Shannon Theorem. For the signals of the EEGs, which have a rather low frequency, 1 msec to 5 msec will be sufficient. The potentials of the EEG signals at the electrodes (or for the electrodes) can be amplified by an analog amplifier before they are AD-converted.
The software performs a check (of the contacts, etc.) for correctness at the beginning of a “session”.
The software controls the respective technical components (air flow, randomized fragrance flow stimuli), shows on the display associated concentration/distraction images/movies, provides acoustic distraction.
The software acquires the resultant EEG signals (as time series), calculates transformed data (FFT etc.) if necessary, extracts relevant quantities (minima, maxima, amplitudes, time spans, . . . ), compares threshold values, categorizes etc.
The software accesses reference data, such as general data on “sound/unsound reaction”, but also individual data (preferably available archive data of the user).
The software combines these values with the temperature values, etc., which have been recorded as well.
The software performs target/actual comparisons of observed versus referenced data.

The “virtual-reality glasses” according to FIG. 3 and the helmet according to FIG. 4 may be provided with “appendages”. The latter include e.g. headphones, EEG leads, a temperature sensor, cable connections between the components and in particular also a tablet or a smartphone, preferably in the glasses attachment, which serves as a “miniaturized PC”. FIG. 7 shows an overview of this.

The consumables (phenylethyl alcohol—PEA, CO₂) and their costs are negligible.

The attachment (e.g. as a helmet according to FIG. 4) would perhaps be used in a person-related manner (repeatedly, like a modern blood pressure monitor that is easy to handle), but it would also be easily sterilizable for multi-person use (e.g. in test centers).

A test could be prepared within a few minutes, and can thus be carried out first before any public step with suspected contact occasion, with a test carried out off-line later on, and a test result reported even more later, with an existing but not great delay in time—compared to today: test+delivery+laboratory+feedback, usually more than two days.

The software checks (via fingerprint, voice recognition, etc.) who the user is, and, if required, creates a forgery-proof documentation of the method steps carried out.

An exemplary representation of a chemosensorily evoked potential (with stimulus onset time of stimulus application, relevant measurement points, etc.) is shown in FIG. 5.

For the generation of chemosensory potentials, the choice of the triggering agent/stimulus allows differentiation and interpretability of the signal obtained. If it is desired to obtain a pure olfactory potential, a substance, such as H₂S or PEA, stimulating exclusively and purposefully the olfactory system can be used. The trigeminal system can specifically be excited with CO₂.

Abbreviations/terms used hereinafter are from English usage: ERP for “event-related potentials”; OERP for olfactory, CSSERP for chemosensory, and tERP for trigeminal ERP.

The classification of ERPs is based on several aspects: according to the 10/20 positioning of the electrodes (e.g. Fz, Cz, Pz, . . . ) at which the signals are recorded, according to the stimulus applied (e.g. acoustic, visual, olfactory), according to the temporal sequence of negative and positive amplitude maxima starting from the stimulus onset, according to the temporal delays of these maxima (latencies), . . .

An olfactometer is used to postulate necessary principles for a standardizable generation of chemosensory stimuli. This tool offers the possibility of embedding a sensory stimulus in a constant, humidified and tempered flow of air.

It makes it possible to let an odorant flow on and off within time windows of less than 20 ms. This as a stimulus in the sense of a pulse. The aim is to realize the change between an odorless and a smellable flow of air without activating mechano- or thermoreceptors of the olfactory mucosa or triggering habituations or adaptations in the event that the increase in the stimulus is not high enough. A time span that is so short is not always necessary, although it is technically possible. Pulse ranges of less than 250 ms are easily possible and can be referred to as pulse compared to the preferably humidified and tempered flow of air that is active for a longer period.

Interstimulation intervals ISI of 30 s to 45 s turned out to be ideal intervals between the stimuli. The choice of the respective interval is determined by the demands of the experiment on the person to be examined.

The flow of air used for presentation should have a flow rate of 7 to 81/min and, because of the concomitant sensitive innervation to be prevented and for better standardizability, it should have a humidity of 70% to 80% and a temperature of 36° C. to 38° C.

The control of the whole experimental setup can take place via a PC and a software developed for this purpose, which is used to define various concentrations and stimulus times as well as correspondingly desired ISI in stimulus classes and to read them out for application. The PC additionally combines in its function the registration of the signals acquired by the EEG amplifier units. They are stored on the PC and evaluated by extended programs.

This allows exact stimuli (of an optical, acoustic or olfactory nature) to be generated in a reproducible manner with regard to start time, duration and intensity and to record the reactions of the user (e.g. by means of EEG devices). The evoked potentials can then be extracted from the repeatedly recorded signals by means of “averaging methods.”

The functionally represented hood according to FIG. 4 or FIG. 7 with the associated components (microprocessor, analog-to-digital converter, control unit and electrodes as well as actuator element, cartridge and virtual-reality glasses and associated headphones) may also be portable, detached from the experimental setup.

Odorous Substances Used

Phenylethyl alcohol (2-phenylethanol, C₈H₁₀O, PEA) is a colorless, liquid and light-sensitive chemical compound from the alcohol group. It occurs naturally in essential oils of hyacinths, peonies, geraniums and numerous other flowers. Being synthetically producible, it is used as a starting material for “sweet” floral fragrances. This substance is considered to be one of the small number of exclusively olfactorily stimulating substances and is therefore particularly suitable for the given issue. CO₂ (carbon dioxide) is a chemical compound of carbon and oxygen. It is a colorless and odorless gas that has been shown to cause selective stimulation of the trigeminal system nasally. In the experimental setup referenced here, concentrations of between 40 vol. %. and 60 vol. %. are used.

An Example of a General Test Procedure

A representation of a chemosensorily evoked potential is shown in FIG. 5. The stimulus onset time of the stimulus application is shown by the vertical arrow, the measurement points shown are possible, but not sole imperative, measurement points.

In a typical session, a simplified 6-channel derivation from the points Fz, Cz, Pz, C3, C4 and from a measurement electrode Fp2 for the acquisition of blink artifacts was, under the aspect of a practicable mode of operation, sufficient for registering the electroencephalographic potential (reference 1).

Unipolar reference points were here electrodes provided on the earlobes and ground electrodes on both sides of the mastoid.

Subsequently, the nose piece (in the example a Teflon tube with a plastic cap and a diameter of 2 mm) was oriented such that, on the one hand, a small amount of residual movability and, on the other hand, a reliable application of the fragrance pulse into the nasal vestibule were accomplished. The nose piece should be about 1.5 cm inside the vestibule. The headphones can improve the noise isolation that has already been mentioned.

All interpretable EPs (optimally 15 individual stimuli to each stimulus class) were then subjected to the averaging procedure (via “averaging”). This allowed the assignment of a specific signal to each stimulus class. Points relevant for the evaluation were measured on each potential curve. For averaging, at least eight single potentials belonging to each stimulus class were required. This in a time span of 480 sec±10%.

At least eight single potentials belonging to each stimulus class can be used for averaging.

The result was a curve with specific positive and negative maxima (compared to FIG. 5). According to the EEG convention, the polarity was named N for an upward and P for a downward deflection of the curve. Regarding their amplitudes and their latency from the time of stimulation, the points P1 (before the negative maximum), the N1 component, and the second maximum positive curve excursion, denoted as P2 in the CSERP nomenclature, were measured. Remark: Different ± orientations of the vertical axis can be found in the literature.

In OERP, P1 is found at about 200 ms to 250 ms, N1 in a time window of 200 ms to 700 ms, and P2 between 300 ms and 800 ms after stimulus onset. In tERP provoked by trigeminal stimulation, these times are shifted about 50 ms to the left on the time axis (references 2, 3).

The clinical relevance of these interactions became clear in studies of patients with olfactory dysfunction who simultaneously exhibited a reduced trigeminal sensitivity. The obvious conclusion was that the olfactory system significantly contributes to a normal function of the trigeminal system (references 4, 5, 6, 7).

The olfactory system significantly contributes to the normal function of the trigeminal system.

The data of psychophysical measurements showed that, when a selective olfactory stimulus was combined with an exclusively trigeminal stimulus, a significant increase in intensity strengths was obtained. The latter were continuous and dependent on PEA and CO₂ concentrations, both as a mixture of substances and when the components were taken into account individually. Increasing concentrations of the additively acting olfactory stimulus evoked a continuous increase in the subjectively perceived stimulus intensity.

These observations held true for both CO₂ concentration levels studied. Hence, the data corroborate those of the previous studies by Kobal and Hummel 1988 as well as by Roscher et al. 1997 and by Livermore et al. 1992. The observations made in the psychophysical measurements were partially corroborated by the results of the electrophysiological studies. The amplitude P2 increased continuously with increasing PEA concentration, although not significantly. The increase in amplitude difference N1P2, however, proved to be significant. Equally significant were, as expected, the differences in amplitudes for the two trigeminal stimulus levels. The dependence of the amplitude size on the concentration of the trigeminal stimulus was thus confirmed.

The potentials evoked are described in the same way for all sensory modalities. However, depending on the respective type of stimulus (acoustic, visual, olfactory, . . . ), the shape and the time profile of the potential differ.

The early and middle potential components are essentially determined by the physical stimulus parameters, such as brightness in the case of a visually evoked potential, and are therefore referred to as exogenous components. They occur in the range of up to approx. 100 msec after stimulus onset. Potential maxima can here be derived from the sensory brain areas that are excited by the specific stimulus. Potential peaks occurring with a (very short) latency of up to approx. 10 msec do not arise in the cortex but in the brainstem and are referred to as brainstem potentials. A rough overview of this is given in FIG. 9.

For acoustically evoked potentials, FIG. 10 shows an example of dependencies between stimulus intensity (sound intensity) and stimulus response.

The same applies to optically triggered stimuli. The visually evoked potentials are highly dependent on technical parameters of the stimulation (monitor, mirror, flash goggles, distance to the eye).

The schematic diagram of FIG. 7 shows technical components of the attachment, though not all of them are needed in combination in every case of use. For example, in EEG sessions in which only visual stimuli are to be analyzed, the “olfactory components” (cartridges, air/fragrance feeders, etc.) may also be absent.

A basic component is similar to a modern bicycle helmet, which may have a weight of less than 200 g. Provided with respective, optionally adjustable contact surfaces, it has high wearing comfort. Several structural variants are possible: closed, grid-shaped, etc., a grid-shaped structure also ensures good ventilation, in addition to a low weight, and, moreover, facilitates any manual readjustment that may be necessary or desired.

These are great advantages compared to the headgear, bands or plastic meshes commonly used today, which are difficult and tedious to put on, often get out of place, cause a sensation of inconvenience, and under which some users will also sweat after a while.

A standardization of electrode positions has existed since the 1950s. However, it has been known for almost as long that in about 20% of all cases the head geometry of the test person deviates very much from the standard, so that a complicated readjustment of electrode placements (i.e. shifting to non-standard positions) will be necessary for obtaining reliable and comparable EEG measurement results, cf. R. W. Homan et al.: Cerebral location of international 10-20 system electrode placement. Electroencephalography and Clinical Neurophysiology, 66, 367 to 382; 1987. Here, too, a helmet will be of advantage: it is comparatively simple to produce different variants according to a fine head size grid, and, moreover, it is also possible to manufacture individual helmets, e.g. by injection molding or 3D printing, so that a “personal helmet” will be created as an attachment, comparable to a “foamed personal ski boot.”

Various materials are possible, including those with which a transparent helmet can be produced, so that the positioning of and the contact pressure at the electrodes can easily be checked with the naked eye, even when the helmet is on.

All potential fluctuations in the EEG registration that do not originate from the brain are regarded as artifacts. Their detection and prevention is one of the most important problems in electroencephalography. The numerous possible artifacts can be divided into person-related or biological artifacts and technical malfunction. The former are caused by the user and are therefore often unavoidable. Technical artifacts are caused by electrode defects, wire defects, general equipment defects or technical external influences. 50 Hz alternating current interference and other electromagnetic interference caused by the ambience (smartphones, laptops, static charges, stroboscopes, olfactometers, . . . ) of the laboratory environment play an important role, cf. “Artefakte in EEG”. S. Zschocke; “Klinische Elektroenzephalographie”, 651 to 685; Springer-Verlag, 2002.

Such interfering influences can be warded off by incorporating electrically conductive materials into suitably designed helmet structures (e.g. as a closed or a honeycomb-shaped shell). The helmet acts as a Faraday cage.

The susceptibility to failure is reduced still further by permanently integrating all wires in a shielded design into the webs or honeycombs of the helmet. As a result, many interference factors that inevitably arise today due to the common “tangled wires” will not even arise.

The arrangement of the electrodes on or in the helmet may correspond to a standard grid, but it may also be based on the individual head shape of the user, as has already been mentioned hereinbefore with respect to the design of various shapes of the helmet.

A further variant of the helmet as an attachment consists of incorporating a respective electrode segment with a grid of possible electrode positions instead of the point positions for the electrodes specified in accordance with the standard. FIG. 8a/b show two examples of such segments. When a helmet is used for the first time, the electrode positions will then first be varied in each electrode segment at the beginning of the EEG session until the optimum positions for the helmet wearer (the user) have been found. These positions are stored as a “head print” so that a new EEG session to be carried out can be started immediately with the ideal positions.

The electrode positions may occupy an ellipsoidal electrode grid or a circular electrode grid, as visualized in FIG. 8a (ellipsoidal) and FIG. 8b (circular).

The electrodes used should have (for the measurement) a mechanical and electrical contact with the scalp that is as permanently stable as possible.

This can be accomplished by rotating or sliding each of the electrodes in their helmet segment holder towards the head surface until sufficient contact has been made.

This may additionally also be indicated by a checking component of the control program disclosed. If the quality of the EEG data to be recorded has to satisfy higher demands, the electrical contact can be improved still further by using electrode gels or electrode pastes suitable for this purpose.

The electrode holder and the region immediately surrounding the same, e.g. grid elements, may be designed to be flexible and thus have a certain pretension, like the grid elements outlined in FIGS. 8a/b. In this way, a permanent, yet to a certain extent still flexible, contact between the electrode and the scalp can be accomplished without any strong sensation of pressure occurring.

The upright diamonds represent, for example, flexible plastic elements that may be incorporated into the helmet segments at the 10/20 positions. The light circles represent possible alternative positions into which an electrode can be inserted or screwed in. A “suitable choice” of the position(s) depends here on a respective individual, real head shape (of the helmet wearer). Once an electrode has been inserted or screwed-in to such an extent that sufficient contact with the head surface has been established, the pretension and/or the flexibility of the plastic element, for example, ensure that the contact between the electrode and the head surface will actually be maintained permanently without the wearer experiencing a strong sensation of pressure.

In many cases of use, scanning can be performed without applying a gel or a paste.

The electrodes are connected to the shielded wires in the helmet and are thus also components in the Faraday cage.

Depending on the respective field of application of the EEG (e.g. scientific, entertainment-oriented), very different variants of attachments may be of interest, which differ in particular with regard to the number of electrodes required.

Among the most frequently used attachments is a “minimal set” that includes the Fz, Cz, and Pz electrodes, which are placed in the middle/crown area of the helmet. In addition, there are further contacts for the A1/A2 leads (earlobes) positioned on lateral segments of the helmet, as well as positions for the Fp1 and Fp2 electrodes, respectively, located on the frontal segment of the helmet and provided for observing eye movements.

Further sensors may be incorporated in the helmet segments, e.g. a temperature sensor for checking or monitoring the test person.

One of the helmet segments may also have attached thereto (by plugging or gluing) a chip, a smart card, a USB stick or the like, with which the EEG signals can be recorded, stored and also passed on directly.

The basic components presented so far, i.e. the helmet and the electrodes-possibly with a “communication chip” and other sensors, represent the minimum version that can be used for EEG recording. The connection to commercially available recording and evaluation devices can be established via wired or wireless interfaces.

The minimal version is particularly suitable for recording sleep EEGs, since in these cases further accessories would be likely to disturb, or for resting EEGs. There are also mobile EEGs as another field of use, since the wearers of the attachment can move freely due to its robustness (inter alia, permanently stable, elastic, mechanical and electrical contact between the electrodes and the scalp; shielding against external interference fields).

For carrying out EEG sessions with other focal points, such as observing reactions to various stimuli, the modular system may be supplemented by further components.

Headphones may fulfil several functions:

Acoustic stimuli can be transmitted very directly to a test person through headphones; additional external (disturbing) devices are then no longer necessary.

In addition, instructions, concerning e.g. the course of the session, can easily (and again undisturbed) be transmitted via headphones, the test person can be reassured, if necessary (nervousness, anxiety), etc.

Headphones can shield the user against unwanted acoustic interference from outside and thus contribute to obtaining artifact-free EEG recordings.

The headphones are integrated on the helmet or easy to attach and also to remove (via a plug/click system). In any case, the necessary wiring is also here provided by means of shielded wires to or in the helmet body.

A “pair of glasses” may comprise various components in different combinations . . .

Depending on the design, visual stimuli of any complexity can be generated by virtual-reality glasses. Again, external devices (PCs, monitors, stroboscopes, etc.) are no longer needed.

In the event that the embodiment in question are smartphone glasses, the display of the device belonging to the user may also, quite individually, be used for display, on the one hand for transmitting visual stimuli, and on the other hand also for optical information, for maintaining vigilance, for entertainment, distraction, etc.

In addition to the fact that it can be used as a display, a tablet T may also be used as a central control and monitoring unit. This means that the complete intelligence of the system is integrated on site.

The same applies to a smartphone.

By means of apps installed thereon, the system becomes self-sufficient from other external components. There are also other advantages: the smartphone can, protected against unauthorized access, store historical data of its owner from previous EEG sessions and directly compare them with currently newly recorded data and draw valid conclusions therefrom. Furthermore, as soon as an EEG “BrainPrint” has been created once and certified, it may serve as a person-related, forgery-proof identification. This identification can be used together with a currently recorded EEG test result for a verified certificate.

The glasses, or more precisely, the complete glasses attachment, may be encased in shielding materials. For example, a pane of transparent EMC glass, which is located between the devices and the wearer's field of vision, is also used for this purpose.

In addition, further sensors may be integrated in the glasses attachment, e.g. for the registration of eye movements, which play an important role in the generation of artifacts in EEG recordings.

For carrying out EEG sessions with olfactory stimuli, it is possible to connect an external olfactometer to the system. Alternatively, the attachment may also be supplemented with suitable hardware and software.

Cartridges (cassettes/containers), which are fixed to the back of the attachment and can be replaced as required, are available in various designs. They contain the odorous substances used, which trigger olfactory stimuli, i.e. for example H₂S and/or PEA, and also CO₂, if the latter should be desired as a “control substance”. The constant flow of air is supported by compressed air contained in another cartridge or, if required, it may also be generated by an air current supplied via a hose connection from a fan or a blower.

Fragrances and air are mixed and enter air/fragrance feeders LDZ, which extend in or on the (lateral) helmet segments and open into the nostrils.

The central control unit (in the “glasses”) controls the temporal and quantitative proportions of the odorous substances via actuators A₁, e.g. in the form of solenoid valves, which are also attached to the helmet.

As with all other components of the system, also the cartridges, air/fragrance feeders, actuators, etc., including their wiring, are designed and installed in such a way that they will not interfere with the EEG recording. For example, potential electromagnetic influences are counteracted by shielding materials, and mechanical noise, e.g. the closing noise of solenoid valves, is muted or played over by the headphones.

The system is a modular system from which, depending on the planned EEG application, the necessary components are assembled, e.g. by means of easily releasable plug-in, screw, click or magnetic connections.

The modular system not only represents a technical device, it also consists of a method for an interference-free reproducible generation and acquisition as well as for an integrated analysis and archiving of multisensory EEG data. This will be explained hereinafter on the basis of fictitious EEG scenarios.

At the beginning of a session or for the first use, a helmet suitable for the person in question will be determined. If it is a first-time use, a helmet will be selected according to the head size or hat size. Then, the electrode positions are set according to a standardized system, checked and, if necessary, adjusted. If the electrode positions have already been stored as a “headprint” in a previous case of use, these settings can be made again immediately. If the test person owns an individual helmet, the adjustment steps can be dispensed with.

A software component—which is either integrated in the helmet chip, accessed via an interface (wired or wireless), or installed, for example, as an app on the smartphone—can check the positions of the electrodes as well as the qualities of the EEG signals received. Any necessary readjustments of the electrodes can be carried out easily or even by the test person's own hand due to the easy accessibility.

Carrying out a resting EEG, a sleep EEG, or a mobile EEG (where the user can move around) does not require any other components apart from the helmet. The acquired EEG signals are stored on the helmet chip, transferred to an external storage medium (laptop, PC, etc.) via an interface, or stored internally (on a tablet or a smartphone).

An analysis or further processing can take place parallel to the acquisition (externally on a laptop, a PC, etc., internally on a tablet or a smartphone) or else with a time delay at any later point in time.

For the recording, the preprocessing as well as the analysis and, if necessary, further processing of the EEG data, there is an abundance of partly freely available (open source) software, for example at MathWorks (de.mathworks.com), especially in the Matlab toolbox (sccn.ucsd.edu/eeglab), at OpenBCI (openbci.com), at LETSWAVE (letswave.org) or at NOCIONS (nocions.org), to name just a few. In addition, the software packages also offer a wide range of special processing methods that are suitable for EEG analyses, (standard) averaging methods; time-frequency analysis (TFA), with Fourier/wavelet transforms; artifact rejection; blind source separation (BSS); spatio-temporal filtering; independent component analysis (ICA); etc.

These processing methods include, in particular, the frequency-amplitude methods.

By adding headphones as a further component, it is possible to carry out EEG sessions in an integrated manner. By auditory stimuli acting on the user, corresponding potentials are generated, and the EEG signals acquired in the process are then analyzed, subjected to further processing, if necessary, and/or archived. Analogous to the possibilities described above, the acoustic stimuli can be generated via a helmet chip, controlled externally by a laptop, PC, etc., or triggered internally by the integrated tablet or smartphone. The above also applies to the analysis of the resultant EEG data.

Advantages of the integrated solution are especially that no further devices are needed and the desired stimulus presentation is thus clearly perceived, in particular also due to the isolation against extrinsic, undesired acoustic influences.

From the abundance of processing methods for the EEG results, the time-frequency-amplitude methods are additionally selected here.

A “pair of glasses” (virtual-reality glasses, VR glasses, smartphone glasses, . . . ) fixed to the front of the helmet can add another component to the system. With a tablet, display or smartphone plugged thereinto, the “pair of glasses” becomes the central control unit of the system and fulfils several functions. It visually shields the user from external environmental influences, it also allows necessary information to be communicated “in writing”, it may be used to entertain a user so as to maintain vigilance e.g. by means of a video game—and it can present visual stimuli.

In addition to these “visible” functions, there are other invisible ones. The “pair of glasses” or the smartphone allows, by means of appropriate software (apps), the control and monitoring of the complete course of an EEG process, in which also other components may be involved, and in particular it also manages the recording, evaluation and archiving of the test results.

It is of great advantage that all recorded data remain on the personal smartphone and—as an additional advantage—can be used as comparative data, which can be compared off-line, at any time during subsequent, further sessions. In many cases, the progress monitoring made possible in this way can at least increase the informative value.

In comparison with the other methods that have already been presented, the execution of EEG sessions, in which the reactions of users to olfactory stimuli are examined, is the most complex one, as regards the required device components and the processes to be controlled.

Via the air/fragrance feeders LDZ provided on or in the helmet, the respective fragrances are added to a continuous flow of air in a program-controlled manner and directed to the nasal opening(s). The technical procedure for measuring the interocular pressure can serve as a guide here.

The system can be supplied with the air flow and the fragrances via an (specially simplified, i.e. modified) olfactometer or via air/gas cartridges integrated on the helmet, which are attached to the back, for example, and thus also act as a physical “counterbalance” to the pair of glasses, among other things.

Preferably, there are three cartridges. One is pressurized as an air reservoir, two additional ones contain exemplarily named odorants. (See the paragraph concerning the odorous substances used).

The cartridges are addressed by a (technical) control unit T via functionally independent actuators A₁ (also actuator elements), which are also integrated in or on the attachment. One of the pressurized cartridges supplies humidified air, another one feeds a first fragrance and the additional one feeds a second fragrance into the flow as a stimulus for the user.

Each of the flows is released by a respective actuator element, collectively controlled by the control unit, which, as hardware or software, may be a component of the pair of glasses.

This control unit can play a key role in the execution of EEG sessions with olfactory stimuli. It tracks the progress of the tests, it controls and triggers, respectively, the air and fragrance flows, it records the resultant EEG signals, it stores the latter internally or transmits them wirelessly or by wire to an external medium.

Another software component makes use of this (technical) control unit for executing various scenarios (sequences of steps) and for analyzing the resultant EEG signals.

References . . .

1. Hummel T, Klimek L, Welge-Lussen A, Wolfensberger G, Gudziol H, Renner B and Kobal G., Chemosensory evoked potentials for clinical diagnosis of olfactory disorders. Hno 48: 481-485, 2000.

2. Hummel T and Kobal G.

Olfactory event-related potentials. In: Methods in chemosensory research, edited by Simon S A, and Nicolelis M A L. Boca Raton: CRC Press, 2001, p. 429-464.

3. Pause B. M. and Krauel K.

Chemosensory event-related potentials (CSERP) as a key to the psychology of odors. Int J Psychophysiol 36: 105-122, 2000.

4. Bensafi M, Frasnelli J, Reden J and Hummel T.

The neural representation of odor is modulated by the presence of a trigeminal stimulus during odor encoding. Clin Neurophysiol 118: 696-701, 2007.

5. Gudziol H, Schubert M and Hummel T.

Decreased trigeminal sensitivity in anosmia. ORL J Otorhinolaryngol Relat Spec 63: 72 to 75, 2001.

6. Hummel T, Knecht M and Kobal G.

Peripherally obtained electrophysiological responses to olfactory stimulation in man: electro-olfactograms exhibit a smaller degree of desensitization compared with subjective intensity estimates. Brain res 717: 160-164, 1996.

7. Husner A, Frasnelli J, Welge-Lussen A, Reiss G, Zahnert T and Hummel T. Loss of trigeminal sensitivity reduces olfactory function. Laryngoscope 116: 1520 to 1522, 2006.

1. An attachment for the head of a person, the attachment coupled to a first control unit (T) and comprising electrodes (E₁ to E₄) for receiving cerebral electrical activity or brain potentials, the electrodes leading to the first control unit (T);
wherein the first control unit (T) has a microprocessor that is capable of recording signals from the electrodes (E₁ to E₄) in a time-related manner via an analog-to-digital converter;
wherein the first control unit (T) is coupled to or actuates an actuator element (A₁) that is capable (LDZ₁) of releasing a fragrance in pulses as a stimulus in a flow from at least one pressurized cartridge (K₁);
wherein the signals of the electrodes (E₁ to E₄) have an EEG signal level in order to record (FIG. 5, FIG. 6) a group of chemosensorically evoked potentials or at least the chemosensorically-influenced temporal sections thereof in a time-related manner and to analyze them—preferably not on the human head.

1a. The attachment according to one of the preceding claims, wherein the material of the attachment and/or of virtual-reality glasses contains electrically conductive components, e.g. wire mesh or sheet metal, for shielding against extrinsic interference fields (Faraday cage).

1b. The attachment according to one of the preceding claims, wherein the first control unit (T) has a microprocessor that repeatedly activates such a stimulus according to a predetermined cycle rate, with respect to one or more of start time, stimulus edge, stimulus concentration and stimulus duration.

1c. The attachment according to one of the preceding claims, wherein a fragrance to be released is suitable for generating a chemosensorily event-related potential by triggering an olfactory or trigeminal stimulus via an air/fragrance feeder (LDZ) (FIG. 7), in particular at a distance of less than one cm from the nasal entrance.

2. The attachment according to one of the preceding claims, wherein the electrodes are arranged in a 10/20 distribution on the attachment, so that, when the attachment is placed on the head of the person, cerebral electrical activity or brain potentials are or can be tapped there and can be transmitted to the analog-to-digital converter and the microprocessor.

3. The attachment according to one of the preceding claims, wherein two earlobe electrodes (A1, A2) are arranged laterally on the attachment for creating one or two reference potentials for the other electrodes and for the reference potential-defined acquisition of the EEG signals from the person as subject or test person.

4. The attachment according to one of the preceding claims, wherein the control unit (T) is coupled to and actuates an olfactometer, in particular to a modified olfactometer integrated in the attachment.

5. The attachment according to one of the preceding claims, wherein the control unit (T) triggers a sensory stimulus with a stimulus edge, embedded in a flow of air, the stimulus being fed with an odorous substance or odorant into the flow of air in a pulsed manner in a time window of less than 250 msec, wherein a steepness of a rise of the stimulus edge is of such a nature that at least 66% of a stimulus concentration are reached within at most 20 msec.

6. The attachment according to one of the preceding claims, in particular claim 5, wherein the stimulations with an odorant arise at intervals of more than 30 sec, in particular less than 45 sec, and the stimulations are repeated at least 8 times at a time interval of 480 sec.

7. The attachment according to one of the preceding claims, wherein an actuator (A₁) is provided as an actuator element that is actuated by the control unit (T) and causes a flow of air with a flow volume of more than 51/min.

8. The attachment according to one of the claims, locally coupled to a monitor used for displaying information that the person wearing the attachment has to follow during the measurement.

9. The attachment according to one of the claims 1 to 7, coupled to virtual-reality glasses used for displaying information that the person wearing the attachment follows.

10. The attachment according to one of the preceding claims, the attachment being a helmet, in particular similar to a bicycle helmet, wherein
in particular the electrodes (E₁ to E₄) in a respective helmet segment holder are movable, especially rotatable or slidable, towards the head surface so far as to have sufficient contact with the head surface, and, in particular, can also be displayed on a display by a checking component of a control program in the microprocessor;
or
the electrodes (E₁ to E₄) are arranged in a respective helmet segment holder under pretension;
or
with helmet segments of the helmet having an electrically conductive material incorporated therein for shielding against extrinsic electromagnetic interference fields (at least one).

11. The attachment according to one of the claims 7 to 10, wherein the actuator (A₁) is connected to a hose piece capable of conducting the flow of air released by the actuator.

12. The attachment according to one of the claims 1 to 11, wherein the microprocessor is provided with a software configured to carry out the execution of various test scenarios—as sequences of test steps—wherein the software separates event-related potentials from a background noise by a summation of at least 8 derived stimulus-synchronous EEG sections, in particular with an “averaging method”.

13. The attachment according to claim 12, wherein the software is configured to perform a check, especially a check of the contacts, for correctness at the beginning of a session or a measurement.

14. The attachment according to one of the preceding claim 12 or 13, wherein the software is configured to control a respective technical component, in particular a flow of air, randomized fragrance flow stimuli, preferably to show on a display associated concentration elements, distraction images or movies, in particular to generate an acoustic distraction.

15. The attachment according to claim 12, wherein the software is configured to acquire resultant EEG signals as time series.

16. The attachment according to claim 12, wherein the software of the microprocessor calculates transformed data, e.g. FFT, extracts relevant quantities, such as minima, maxima, amplitudes, time spans, and/or compares threshold values or carries out categorizations.

17. The attachment according to claim 12, wherein the software of the microprocessor is configured to access reference data, in particular general data on stimulus-adequate reactions, or individual data, such as archive data.

18. The attachment according to claim 17, wherein the software combines these values with temperature values that have been acquired as well.

19. The attachment according to claim 17, wherein the software is configured to perform target/actual comparisons with respect to observed vs. referenced data, in particular to decide offline whether a result that is not obtained on humans and not on animals is positive/negative.

20. A method for an interference-free reproducible generation of multisensory EEG data, in particular olfactory-dependent signal partitions of an EEG.

21. The use or usability of the attachment according to one of the claims 1 to 19 for an off-line test, with measurements from the attachment and, spatially and temporally remote therefrom, a test with results.

22. gap

23. gap

24. An attachment for the head of a person, the attachment coupled to a control unit (T), virtual-reality glasses and headphones, and
comprising electrodes (E₂ to E₄) in a respective helmet segment holder for receiving cerebral electrical activity or brain potentials, the electrodes leading to the first control unit (T);
wherein the first control unit has a microprocessor that is capable of recording signals from the electrodes (E₁ to E₄) in a time-related manner via an analog-to-digital converter;
wherein the first control unit (T) is coupled to an actuator element (A₁) that is capable of releasing a flow of humidified air from a pressurized first cartridge (K₁) and capable of feeding, separately therefrom, a pulsed fragrance as a stimulus into the flow of air;
wherein the signals of the electrodes have an EEG signal level in order to record (FIG. 5, FIG. 6) a group of chemosensorically evoked potentials or at least the chemosensorically-influenced temporal sections thereof in a time-related manner and to analyze them—preferably not on the human head.

24a. The attachment according to claim 24 with one of claims 1a, 1b or 1c, without a respective reference to claim 1.

25. The attachment according to claim 24, wherein the control unit (T) is configured to trigger a sensory stimulus embedded in a flow of air, the stimulus being feedable with an odorous substance or odorant into the flow of air in a pulsed manner in a time window of less than 250 msec.

26. The attachment according to one of the preceding claim 24 or 25, wherein the control unit (T) is coupled to an olfactometer and actuates the same.

27. The attachment according to claim 26, wherein the control unit (T) is coupled to a modified olfactometer integrated in the attachment.
